(19) United States
(12) Patent Application Publication
(10) Pub. No.: US 2012/0203204 A1
(43) Pub. Date: Aug. 9, 2012

(54) Retro-Primed Medication Delivery System

(76) Inventor: David M. Briggs, Morristown, TN (US)

(21) Appl. No.: 13/242,612

(22) Filed: Sep. 23, 2011

Related U.S. Application Data
(63) Continuation-in-part of application No. 13/023,261, filed on Feb. 8, 2011.

(51) Int. Cl. A61M 5/14

(52) U.S. Cl. 604/518; 604/83

(57) ABSTRACT

A system and method for intravenous, intramuscular, or subcutaneous delivery of a reconstituted medication fluid with simultaneous, uninterrupted delivery of a primary fluid. A medication fluid is prepared by reconstituting a medication with a primary fluid as diluent. A container adapted to receive a medication is retro-primed with primary fluid from a primary bag. The primary fluid mixes with the medication to reconstitute the medication and form a medication fluid. A medication delivery system and method according to the present invention allows for the continuous infusion of primary fluid both during and after the administration of a reconstituted medication, using a closed system with reduced opportunities for contamination and infection.

### CROSS-REFERENCE TO RELATED APPLICATIONS

This Application is a continuation-in-part of U.S. patent application Ser. No. 13/023,261, filed Feb. 8, 2011.

### STATEMENT REGARDING FEDERALLY SPONSORED RESEARCH OR DEVELOPMENT

Not Applicable

### BACKGROUND OF THE INVENTION

1. Field of Invention

The present invention relates to systems and methods for the introduction of fluids and particularly to systems and methods for delivering medications by intravenous, subcutaneous, or intramuscular introduction.

2. Description of the Related Art

It is common practice to administer many medications in fluid form through intravenous, subcutaneous, or intramuscular introduction. In many cases, a medication is supplied from its manufacturer as a powder or fine solid. Before a powdered medication can be administered to the patient in an intravenous, subcutaneous, or intramuscular manner, the powdered medication must be mixed with a liquid (or diluent) to prepare a fluid (hereinafter “medication fluid”). In other cases, a medication is supplied from its manufacturer as a liquid premix forming a pre-prepared medication fluid; nevertheless, in almost all such cases, the liquid medication from the manufacturer must still be diluted before it can safely be administered to a patient in an intravenous, intramuscular, or subcutaneous manner.

Herein, the term “reconstitution” is used to describe both the process of diluting a powdered medication and the process of mixing a liquid medication with another fluid; in both processes, the original concentrated medication is reconstituted into a medication fluid. Additionally, while much of the subsequent discussion relates to the intravenous administration of reconstituted medications, persons of skill in the art will recognize that much of the below discussion is also applicable to situations in which a medication is administered intramuscularly or subcutaneously.

Many treatment regimes require that a patient receive, over an extended period of several hours, a fluid such as a saline solution or a dextrose solution. These fluids (collectively referred to as “primary fluids” herein) are used to treat a number of symptoms that present in a wide variety of medical conditions—symptoms such as dehydration or hypoglycemia.

When a patient must receive both a primary fluid and a reconstituted medication fluid as part of a treatment regimen, a potential conflict arises. Clinicians and other medical practitioners would like to find a way to administer the reconstituted medication fluid without interrupting the steady delivery of the primary fluid. At the same time, it is important in some medical treatments that a reconstituted medication not be introduced to a patient's system too quickly. Thus, the need to administer the reconstituted medication fluid and the need to ensure the uninterrupted delivery of the primary fluid superficially present competitive dosage regimens.

Practitioners of the medical arts are familiar with a number of systems and methods that attempt to reconcile the competing treatment requirements described above. Each of these systems and methods has drawbacks, which are described below.

One method of administering a medication intravenously involves using a syringe; a closely related method involves using a syringe pump. With a syringe, a clinician slowly pushes the medication from the syringe into the patient's intravenous line (“IV line”). This manual push process requires considerable time, as the clinician must remain at the patient's bedside until the entire medication dose has been administered. There exists the possibility, even likelihood, that a clinician will push the medication too quickly, resulting in pain or other complications for the patient. When a syringe pump is used (with the syringe pump essentially assuming the clinician's role of pushing the medication fluid, thereby allowing the clinician to leave the patient's bedside), the clinician must ensure that the syringe pump is compatible with the syringe being used. A hospital or treatment facility may have to have many different syringe pumps in order to accommodate syringes of different sizes and styles, multiplying expenses. Further, a clinician must be careful that the syringe pump is calibrated to push the medication fluid at the proper rate to ensure the medication's effective agency and to avoid pain and complications for the patient. The calibration of the syringe pump presents an opportunity for the introduction of error. Moreover, whether manually pushing the medication fluid or using a syringe pump, the syringe usually has a limited fluid volume, which may accommodate less fluid volume than is recommended for the medication being administered. For some treatments, it is advisable to dilute a medication dose to a greater extent than is possible when using a syringe for intravenous administration.

Another technique for administering a medication fluid involves the use a bag or container (generally “bag”) with the medication “pre-mixed”—i.e., the medication is already mixed with the fluid before the bag is brought to the patient's bedside. Normally, pre-mixed bags containing medication fluids (hereinafter “pre-mixed IV medication bags”) must be mixed in a clean room, generally in accordance with Chapter 797 guidelines issued by the U.S. Pharmacopia (hereinafter “797 guidelines”). It is expensive and difficult for hospitals to maintain a sterile, 797-compliant pharmacy clean room on site for mixing medication fluids. Some hospitals utilize robotic medication admixture systems, like the system described in U.S. Pat. No. 7,783,383, to reconstitute medications in a sterile, 797-compliant environment; however, such robotic systems are expensive, and any machine errors made by such a system may be perpetuated through a large number of prepared medication fluids. Therefore, pre-mixed IV medication bags are often prepared at a central mixing location, frozen, and then distributed in cold storage transportation to hospitals, where the bags remain frozen until shortly before use. However, once the pre-mixed IV medication bag is thawed, the pre-mixed IV medication bag has a limited shelf life, even when kept ready for use under refrigeration. Further, space and cost considerations limit the number and variety of liquid pre-mixed IV medication bags, whether frozen or refrigerated, that a hospital can keep on site. Thus, the available selection at a given hospital of pre-mixed IV medication bags for a particular medication may not include the specific volume of fluid or concentration of medication that a physician requires for a particular patient.

Another technique for administering a medication fluid involves using a secondary “piggyback” system, in which a bag with powdered medication is mixed with secondary fluid before the bag is brought of the patient's bedside. Again, under normal circumstances, secondary piggyback bags containing medication fluids (“secondary piggyback IV medication bags”) must be mixed in a clean room, generally in accordance with 797 guidelines. It is expensive and difficult for hospitals to maintain a sterile, 797-compliant pharmacy clean room on site for mixing medication fluids. Therefore, secondary piggyback IV medication bags are often prepared at a central mixing location, refrigerated, and then distributed in refrigerated transportation to hospitals, where the bags remain refrigerated until shortly before use. However, once the powdered medication is mixed with the fluid, secondary piggyback IV medication bags has a limited shelf life, even when kept ready for use under refrigeration. Further, space and cost considerations limit the number and variety of secondary piggyback IV medication bags that a hospital can keep on site. Thus, the available selection at a given hospital of secondary piggyback IV medication bags for a particular medication may not include the specific volume of fluid or concentration of medication that a physician wants or prescribes for a particular patient.

Another system for simultaneously administering a primary fluid and a reconstituted medication involves the use of a burette. FIG. 1 illustrates one example of this delivery system. The system includes a primary bag 110 filled with a primary fluid, such as a saline solution or a dextrose solution. The primary bag 110 includes an IV administration port 112, through which the primary bag is connected to other components of the intravenous delivery system; the primary bag 110 also includes a medication port 114, which includes an access membrane through which material may be inserted into the primary bag 110. A spiked cannula 115 penetrates the access membrane of the IV administration port 112 of the primary bag 110, thereby allowing fluid to flow from the primary bag 110 through the cannula 115 into a rigid burette chamber 116. The burrette chamber includes graduated markings 124 to allow a clinician to observe and measure the fluid levels within the burrette chamber 116. Fluid flows from the burette chamber 116 through a drip chamber 130 into a line 133. The line 133 is connected to a connector 140 for connecting the system to an IV catheter. In the illustrated embodiment, the line 133 includes other components, including a slide clamp 134 and a roller clamp 136 for regulating fluid flow through the line 133, and injection sites 138 comprising devices deigned to be accessed by a small-scale fluid fittings such as, for example, Luer-Lok® fittings by Becton Dickinson. (Hereinafter such an injection site device will be referred to as “Luer-activated device” or LAD, and the fittings adapted to cooperate with such as device shall be called “Luer lock connectors.”)

In this prior art setup, medications are added to the primary fluid through an LAD injection site 118 in the burette chamber 116. The burette chamber 116 also includes an air vent 120 with a clamp 122; in order for fluid to evacuate the rigid burette chamber 116, it is necessary for air to be able to fill the chamber. This air vent 120, by allowing outside air to enter the burette chamber 116, presents an infection risk, as contaminants and airborne microorganisms may enter the burrette chamber 116 through air vent 120, therein mixing with the primary fluid and traveling with the fluid to the patient. Further, once the burrette chamber 116 has emptied of fluid, or is nearly empty, there is a risk that an air bubble will enter the line 133 and pass into the patient, with adverse consequences. Therefore, it is often necessary for a clinician to monitor the burette system closely and then close off the flow of fluid from burette chamber 116 before all of the fluid in the burette chamber 116 has emptied the chamber 116. Thus, in order to avert the possibility of an air embolism, some of the medication mixed in the fluid often is not delivered to the patient. Further, when using a burette delivery system in combination with an IV, if the flow of fluid through the IV ceases, clotting may occur, with attendant complications. If the IV line is a central line, and the IV must be restarted, then the cost to restart the central line IV could be considerable.

Due to the drawbacks in the existing systems and methods for simultaneously administering a primary fluid and a reconstituted medication fluid, a need exists for a different approach.

### BRIEF SUMMARY OF THE INVENTION

Described herein is a system and method for preparing a medication fluid by reconstituting a medication with a primary fluid as diluent. A medication delivery system and method according to the present invention allows for the continuous infusion of primary fluid both during and after the administration of a reconstituted medication fluid, using a closed system with reduced opportunities for contamination and infection.

The present invention uses a combination of flexible fluid containers (hereinafter “bags”) to reconstitute a medication with primary fluid and deliver it to a patient. The retro-primed medication delivery system includes a “primary bag” containing a primary fluid, such as a saline solution or a dextrose solution, and a secondary bag (or “medication bag”) adapted to hold a medication. The primary bag is generally a large volume parenteral bag. The primary bag and the secondary or medication bag are connected to a fluid transfer system in fluid communication with a delivery device for passing fluid to the patient (such as an IV catheter); the fluid transfer system (for example, an IV administration set) allows the passage of fluid between the primary bag, the medication bag, and the delivery device. The method involves “retro-priming” the medication bag with primary fluid from the primary bag before beginning administration of a reconstituted medication fluid from the medication bag to the patient. Persons skilled in the art will recognize that, although the following discussion focuses primarily on the use of the present invention for intravenous delivery of reconstituted medications, the present invention is capable of being used in other contexts, including in intramuscular and subcutaneous administration.

During use of the retro-primed medication delivery system, the primary bag and the medication bag are both connected to the fluid transfer system. The primary bag is filled with a primary fluid, such as a saline solution or a dextrose solution. The medication bag is lowered into a position such that primary fluid from the primary bag flows through the fluid transfer system into the medication bag (thereby “retro-priming” the medication bag with fluid from the primary bag). The primary fluid flowing into the medication bag mixes with medication in the bag to form a medication fluid. In some embodiments, the medication bag arrives at the bed-side with the bag already containing a powdered medication; this powdered medication is then reconstituted with the primary fluid from the primary bag. In some embodiments, the medication comprises either a powdered drug or a concentrated liquid medication supplied from a manufacturer in a vial; in these embodiments, the medication bag includes a vial adapter configured to access the vial and allow the medication to pass from the vial into the medication bag, where it is diluted with the primary fluid from the primary bag. In some embodiments, after the medication bag has been retro-primed with primary fluid, a liquid medication is added to the bag by injecting the medication from a syringe into an injection site on the medication bag. In all of these embodiments, a medication is mixed with primary fluid within the medication bag, the primary fluid having been supplied from the primary bag. The medication bag includes graduated markings, visible from the outside of the bag, to allow a clinician using the bag to determine how much fluid has been added to the bag. Once the medication bag has been retro-primed and the medication mixed with the primary fluid to form the medication fluid, the medication bag is moved into a position such that the medication fluid from the medication bag flows through the fluid transfer system and the delivery device into the patient, with the medication fluid taking precedence over the primary fluid from the primary bag until such time as all of the medication fluid has been administered. In this way, while the reconstituted or diluted medication is being delivered to the patient, the patient continues to receive primary fluid, as the medication fluid includes the primary fluid that is part of the patient's treatment regimen. Thus, the present system and method allow for the administration of a reconstituted or diluted medication together with the uninterrupted administration of a primary fluid.

### BRIEF DESCRIPTION OF THE SEVERAL VIEWS OF THE DRAWINGS

The above-mentioned features of the invention will become more clearly understood from the following detailed description of the invention read together with the drawings in which:

FIG. 1 is a perspective view of one example of a prior art setup;

FIG. 2a is a perspective view of one embodiment of the present invention;

FIG. 2b is an exploded view of the embodiment shown in FIG. 2a;

FIG. 3a is a perspective view of a step in a method of using the embodiment shown in FIGS. 2a and 2b;

FIG. 3b is a perspective view of a subsequent step in the method;

FIG. 3c is a perspective view of a subsequent step in the method;

FIG. 3d is a perspective view of a subsequent step in the method;

FIG. 4 is a perspective view of one embodiment of the present invention;

FIG. 5 is a perspective view of one embodiment of the present invention;

FIG. 6 is a perspective view of another example embodiment of the present invention, including a medication bottle that is retro-primed from the primary bag;

FIG. 7 is a perspective view of another example embodiment of the present invention, including a dual-chambered bag with an upper chamber including medication; and

FIG. 8 is a perspective view of another example embodiment of the present invention, including a dual-chambered bag with a side-chamber including medication.

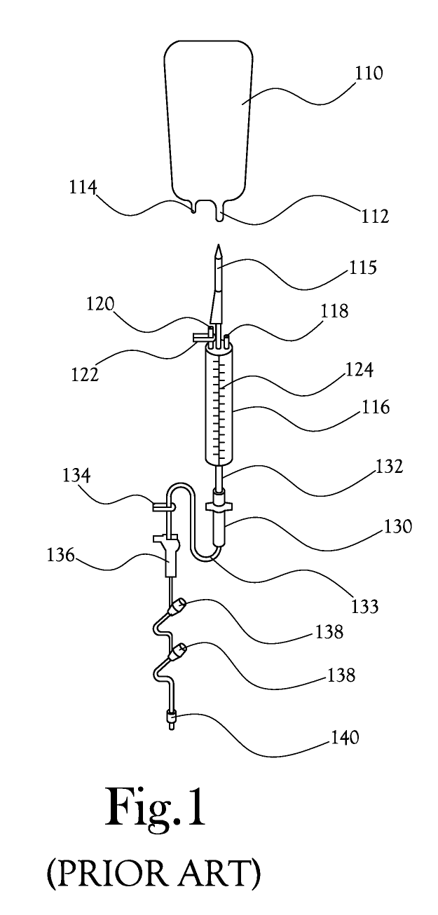

### DETAILED DESCRIPTION OF THE INVENTION

Disclosed herein is a system and method for intravenous (IV) delivery of a reconstituted medication with simultaneous, uninterrupted delivery of a primary fluid. The retro-primed medication delivery system includes a primary bag containing a primary fluid, such as a saline solution or a dextrose solution, possibly with additives such as potassium chloride, and a secondary bag (or “medication bag”) adapted to hold a medication. The primary bag and the secondary or medication bag are connected to a fluid transfer system in fluid communication with a delivery device for passing fluid to the patient (such as an IV catheter); the fluid transfer system allows the passage of fluid between the primary bag, the medication bag, and the delivery device. The method involves “retro-priming” the medication bag with primary fluid from the primary bag before, adding a medication if it is not already present, thoroughly mixing the medication with primary fluid in the medication bag, and beginning administration of the now reconstituted medication fluid from the medication bag to the patient.

During use of the retro-primed medication delivery system, the primary bag and the medication bag are both connected to the fluid transfer system. The primary bag is filled with a primary fluid, such as a saline solution or a dextrose solution. The medication bag is lowered into a position such that primary fluid from the primary bag flows through the fluid transfer system into the medication bag (thereby “retro-priming” the medication bag with fluid from the primary bag). The primary fluid flowing into the medication bag mixes with medication in the bag to form a medication fluid.

In some embodiments, the medication bag arrives at the patient's bed-side with the bag already containing a powdered medication; this powdered medication is then reconstituted with fluid sourced from the primary bag. In some embodiments, the medication bag arrives at the patient's bedside with the bag already containing a concentrated liquid medication; this liquid medication is then diluted and mixed with fluid sourced from the primary bag. In some embodiments, the medication comprises a powdered medication supplied from a manufacturer in a vial; in these embodiments, the medication bag includes a vial adapter configured to access the vial, allow fluid from the medication bag to pass into the vial and mix with the powdered medication in a liquid suspension, thereby reconstituting the medication, and then allow the reconstituted medication fluid to pass into the medication bag. In some embodiments, the medication comprises a concentrated liquid medication supplied from a manufacturer in a vial; in these embodiments, the medication bag includes a vial adapter configured to access the vial and allow the liquid medication to pass from the vial into the medication bag, where it is diluted with the primary fluid from the primary bag. In some embodiments, after the medication bag has been retro-primed with primary fluid, a liquid medication is added to the bag by injecting the medication into an injection site on the medication bag.

In all of these embodiments, a medication is mixed with primary fluid within the medication bag, the primary fluid having been sourced from the primary bag. The medication bag includes graduated markings, visible from the outside of the bag, to allow a clinician using the bag to determine how much fluid has been added to the bag. Once the medication bag has been retro-primed and the medication mixed with the primary fluid to form the medication fluid, the medication bag is moved into a position such that primary fluid is no longer flowing into the medication bag and the medication fluid from the medication bag flows through the fluid transfer system and the delivery device into the patient, with the medication fluid taking gravimetric precedence over the primary fluid from the primary bag until such time as all of the medication fluid has been administered. In this way, while the reconstituted or diluted medication is being delivered to the patient, the patient continues to receive primary fluid, as the medication fluid includes the primary fluid that is part of the patient's treatment regimen. Because the medication fluid from the secondary bag is sourced with primary fluid from the primary bag, the primary fluid continues to be administered to the patient even though the flow of fluid directly from the primary bag is halted during the time in which the medication fluid from the secondary bag is being administered. Thus, after administering the medication fluid, there is no need for a clinician to engage in “fluid catch-up” with the primary fluid.

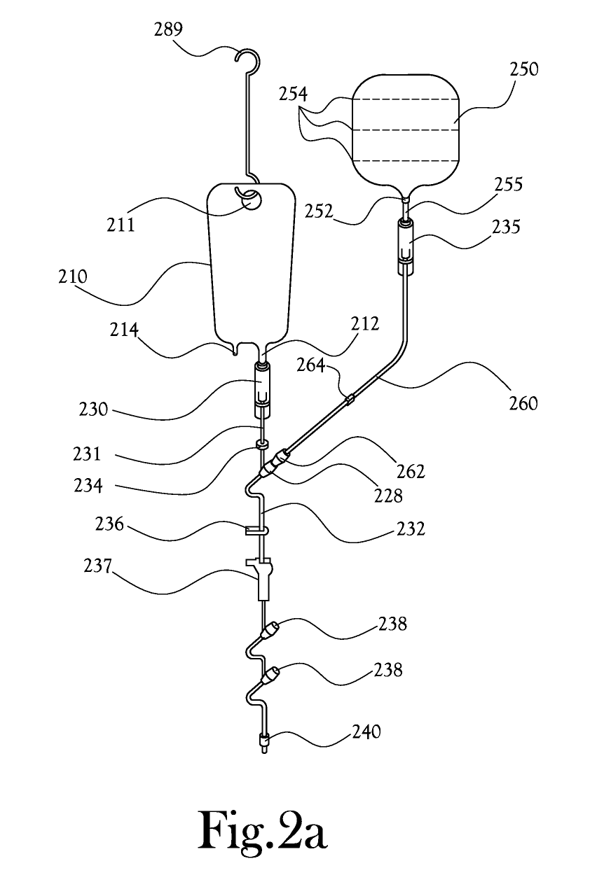

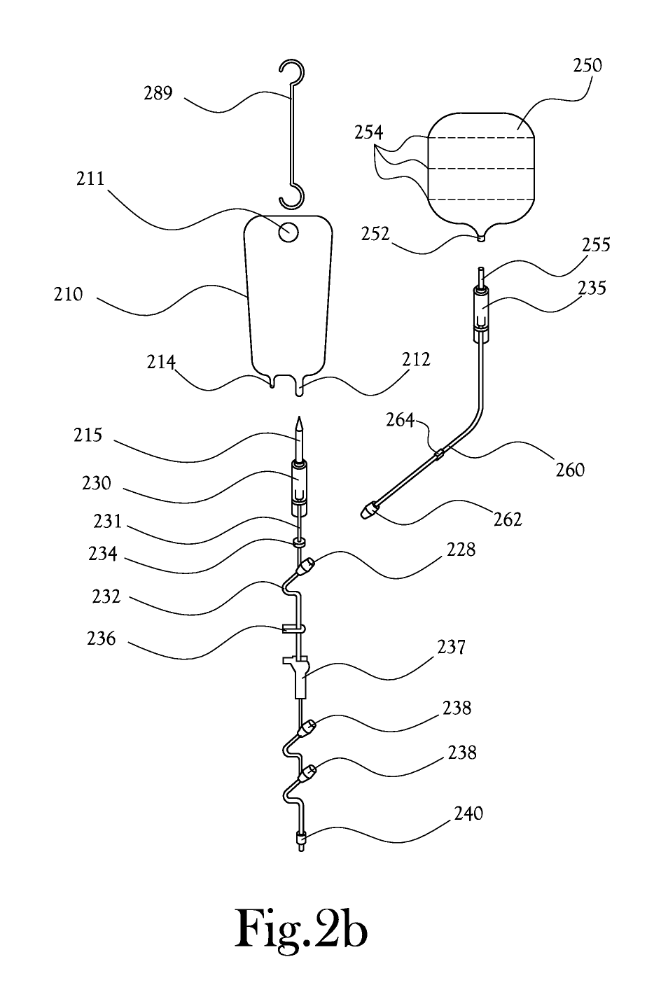

One embodiment of a reconstituted medication delivery system according to the present invention is illustrated in FIG. 2a. As shown in FIG. 2a and in the exploded view of the same embodiment in FIG. 2b, the system includes a primary bag 210 and a medication bag 250. In the illustrated embodiment, the medication bag 250 is supplied pre-filled with a powdered medication. The primary bag 210 includes an IV administration port 212, through which the primary bag is connected to other components of the intravenous delivery system, and an access medication port 214. The medication bag 250 includes its own IV administration port 251. Both of the IV administration ports 212 and 251 include access membranes which a hypodermic needle, spiked cannula or similar device can penetrate to gain access to the interior of the bag. In the illustrated embodiment, the primary bag 210 includes a handle 211, which can be used with a hanger 289 to suspend the primary bag 210 from other equipment.

In the illustrated embodiment, a spiked cannula 215 penetrates the access membrane of the IV administration port 212 of the primary bag 210, thereby allowing fluid to flow from the primary bag 210 through the cannula 215 into a drip chamber 230. Fluid flows from the drip chamber through a primary line 231 toward a principal line 232; the primary line 231 intersects the principal line 232 near a primary LAD injection site 228. In the illustrated embodiment, the primary line 231 includes a check valve 234 to prevent fluid from washing back toward the drip chamber 230 and the primary bag 210.

The principal line 232 and primary line 231 are part of a larger IV administration set, which also includes a medication line 260, which is in fluid communication with a medication line 260 through the primary LAD injection site 228 and a Luer lock connector 262. The other end of the medication line 260 is connected to a drip chamber 235, which is connected to a spiked cannula 255 for penetrating the IV administration port 252 of the medication bag 250. The principal line 232 is connected to a Luer lock connector 240 for connecting the system to an IV catheter. In some embodiments, the principal line 232 includes other pieces, such as a slide clamp 236 and a roller clamp 237 for regulating fluid flow through the principal line 232, or additional LAD injection sites 238.

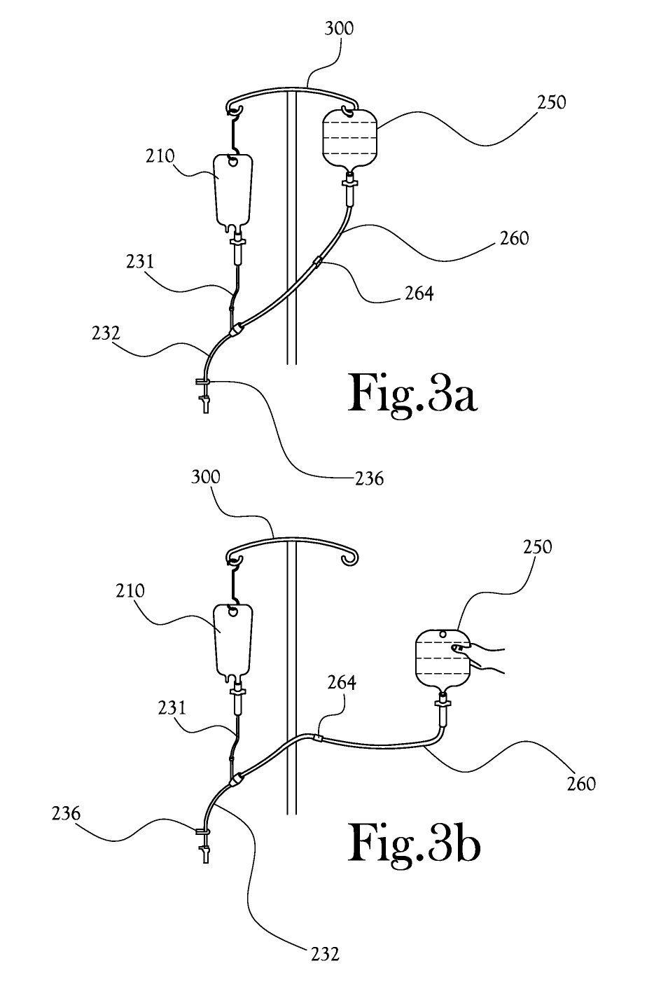

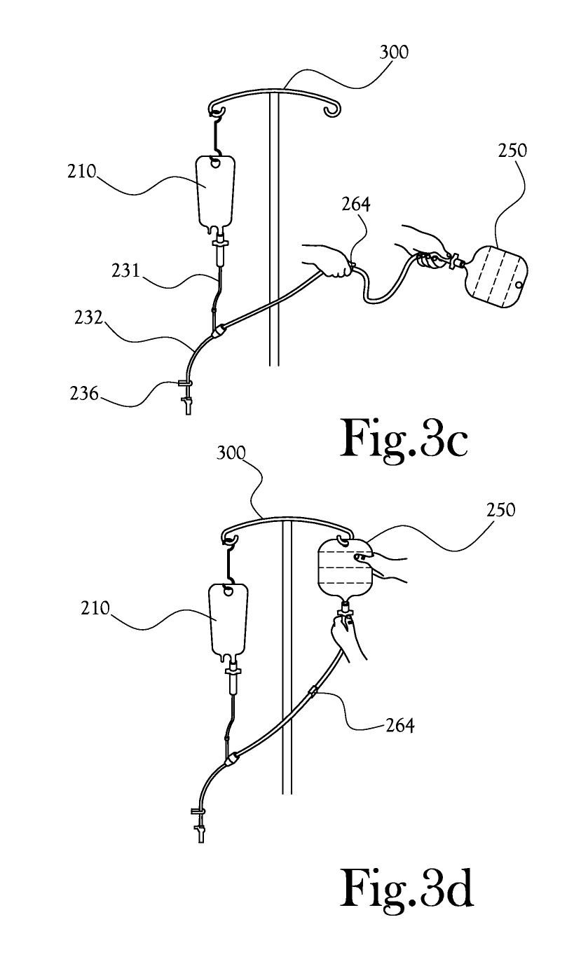

FIGS. 3a through 3d illustrate a method of using the system described in FIGS. 2a and 2b to reconstitute a medication with fluid from the primary bag, thereby retro-priming the medication. The primary bag 210 and the medication bag 250 are both connected to their respective lines 231 and 260, as described above. Initially, both bags are suspended from a hospital pole 300, as seen in FIG. 3a. As stated above, the medication bag 250 is pre-filled with a medication in powder form. As shown in FIGS. 3b and 3c, the medication bag 250 is lowered so that it is at a lower level than the primary bag 210. A clamp 236 on the principal line is closed, and a clamp on the medication line 260 is opened, allowing primary fluid from the primary bag 210 to flow through the medication line 260 into the medication bag 250, where the primary fluid mixes with the medication to reconstitute the medication and form a medication fluid. The medication bag 250 is filled with fluid to a predetermined desired level; in some embodiments, the medication bag 250 includes graduated markings on the surface of the bag to allow a clinician to observe and measure how much fluid has filled the medication bag 250. After the medication bag 250 has been filled with fluid to the desired level, the medication bag 250 is hung from the pole 300 at a higher elevation than the primary bag 210, as shown in FIG. 3d.

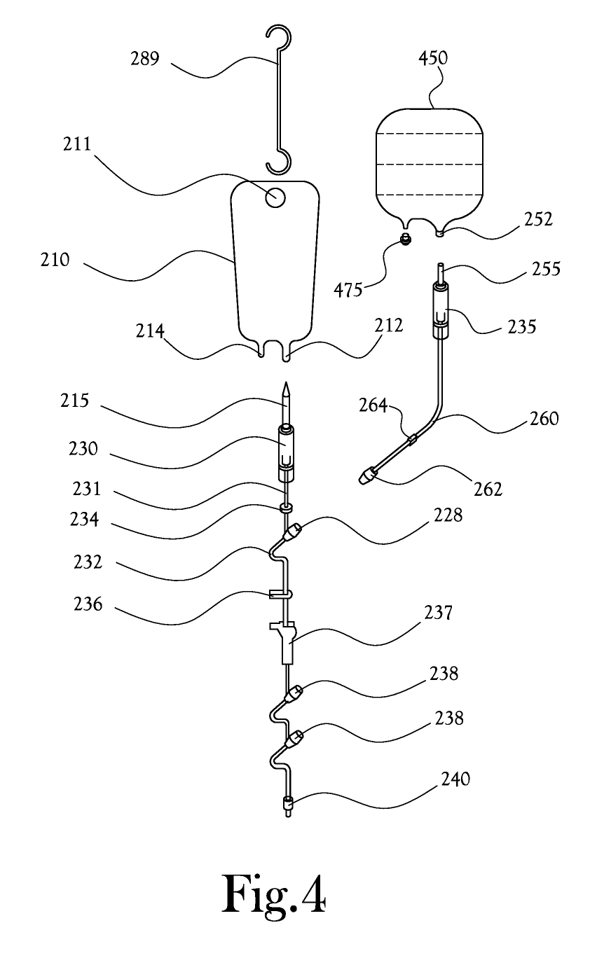

Another embodiment of the present invention is illustrated in FIG. 4. Most of the components of this system are identical to the components illustrated in FIGS. 2a and 2b, but in this embodiment the medication bag 450 includes a Luer lock connector needleless injection site 475. In this embodiment, the initially empty medication bag 450 is first retro-primed with primary fluid from the primary bag 210, as seen with in method illustrated in FIGS. 3a through 3d, filling the medication bag 450 to a desired fluid volume level. Then, before the medication bag 450 is moved into a position elevated above the primary bag, the clamp 264 on the medication line 260 is closed, and then a medication is added through the Luer lock connector needleless injection site 475. In some embodiments, the addition of the medication to the medication bag 450 involves filling a syringe with a concentrated medication in liquid form, connecting the syringe to the Luer lock connector needleless injection site 475, and pushing the medication from the syringe into the medication bag 450. The medication bag 450 is then shaken to effect thorough mixing of the medication and the sourced primary fluid.

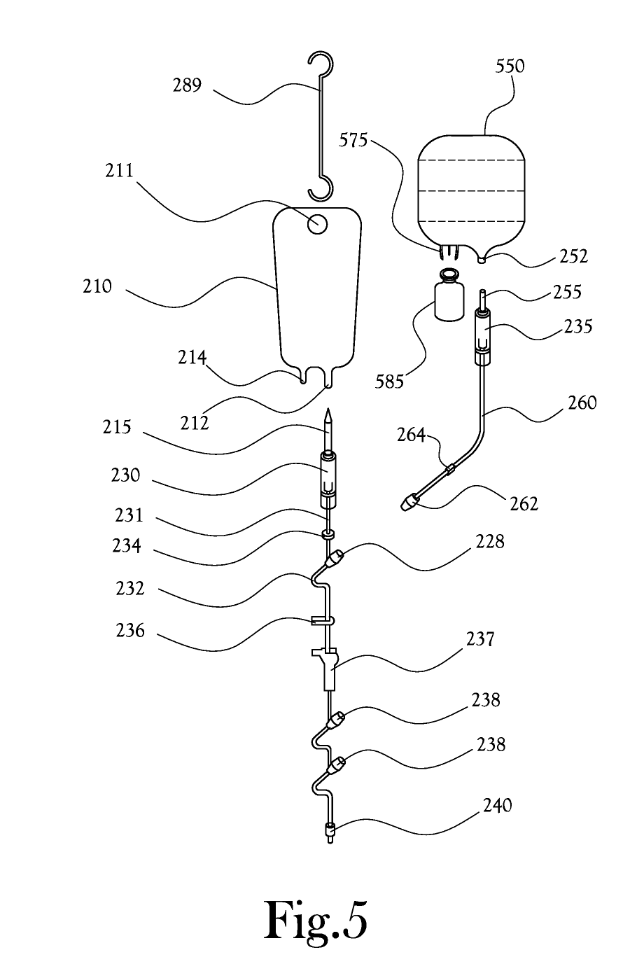

Another embodiment of the present invention is illustrated in FIG. 5. Most of the components of this system are identical to the components illustrated in FIGS. 2a and 2b, but in this embodiment the medication bag 550 includes a vial adaptor 575 configured to receive a vial 585. Many powdered and liquid medications are supplied to hospitals and medical providers in small vials, including unit dose vials. In this embodiment, the initially empty medication bag 550 is first retro-primed with primary fluid from the primary bag 210, as seen with the method illustrated in FIGS. 3a through 3d, filling the medication bag 550 to a desired fluid volume level. Then, the clamp 264 on the medication line 260 is closed, and a medication is added through the vial adaptor 575 on the medication bag 550. In some embodiments, the vial adapter 575 includes a spiked cannula adapted to perforate an access membrane on the cap of a vial. In those embodiments, after the medication bag 550 has been filled with primary fluid, the vial 585 is connected to the vial adapter 575, the spiked cannula of the vial adapter perforates the access membrane in the cap of the vial, allowing medication to move from the vial into the medication bag 550.

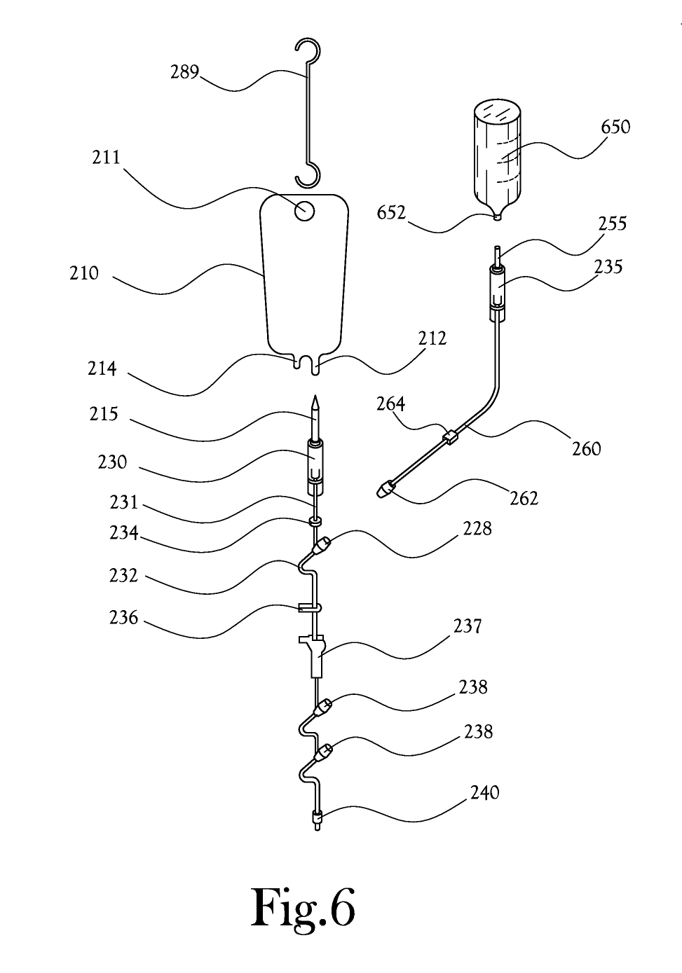

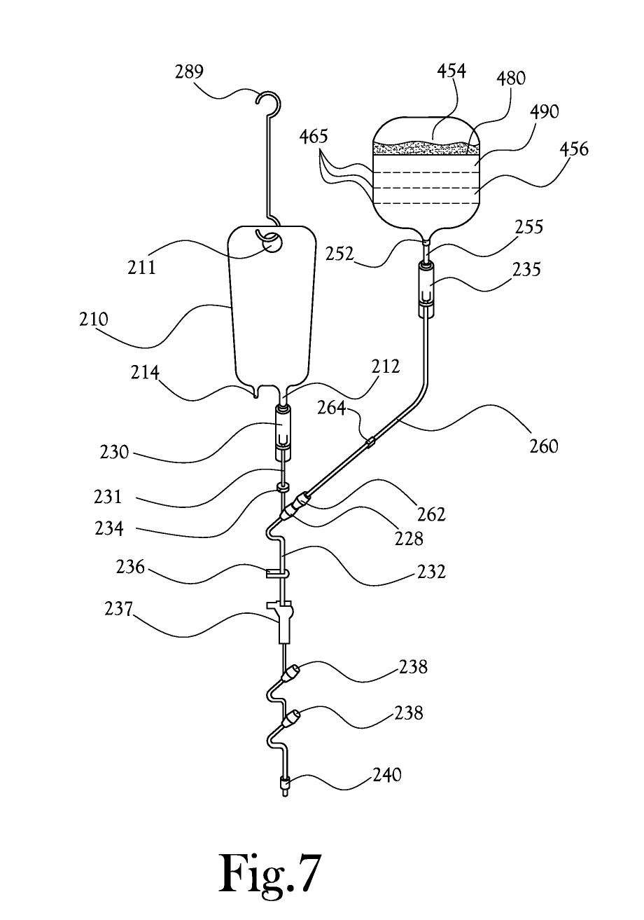

Another embodiment of the present invention is illustrated in FIG. 6. Most of the components of this system are identical to the components illustrated in FIGS. 2a and 2b, but in this embodiment the medication bag is replaced by a rigid or substantially rigid medication bottle 650, which includes either a standard bottle stopper or adapter 652 to receive a spiked cannula 255 to put the interior of the medication bottle 650 in fluid communication with the dual vent secondary medication set or line 260. In this embodiment, the medication bottle 650 is retro-primed with primary fluid from the primary bag 210, as seen with the method illustrated in FIGS. 3a through 3d, filling the medication bottle 650 to a desired fluid volume level. In some embodiments, the medication bottle 650 if filled with medication before the medication bottle 650 is connected to the setup; in other embodiments, after the medication bottle 650 has been filled with primary fluid to the desired fluid volume, the medication bottle can be injected with medication from a syringe and an access device as needed into the medication bottle 650. In some embodiments, the medication bottle 650 includes pre-marked lines for spotting fluid levels within the medication bottle.

Another embodiment of the present invention is illustrated in FIG. 7. Most of the components of this system are identical to the components illustrated in FIGS. 2a and 2b, but in this embodiment the medication bag is replaced by a Dual Chambered Bag 490, which includes an adapter 252 to receive a spiked cannula 255 to put the interior of the dual chambered bag 490 in fluid communication with the secondary medication set or line 260. In this embodiment, the Dual Chambered Bag 490 includes an upper chamber 454 containing medication or concentrated drug and a lower chamber 456; the two chambers are separated by a Horizontal Separation Seal 480. The Dual Chambered Bag 490 is retro-primed with primary fluid from the Primary Bag 210, as seen with the method illustrated in FIGS. 3a through 3d, filling the lower chamber 456 of the Dual Chambered Bag 490 to a desired fluid volume level as labeled by a range of graduated markings 465 beginning with a minimum of fluid required to allow equally applied pressure to force the Horizontal Separation Seal 480 open to mix the retro primed diluent in the lower chamber 456 and the concentrated drug from the upper chamber 454 together for desired mixing. In some embodiments, the Dual Chambered Bag 490 has the upper chamber 454 filled with powdered antibiotic medication, and others have the upper chamber 454 filled with medication in a liquid concentrate.

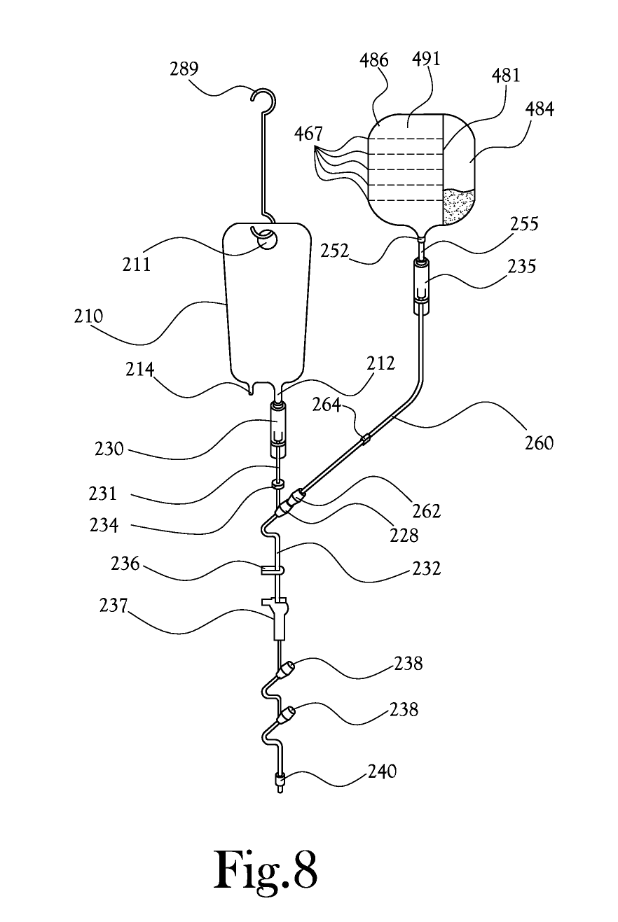

Another embodiment of the present invention is illustrated in FIG. 8. Most of the components of this system are identical to the components illustrated in FIGS. 2a and 2b, but in this embodiment the medication bag is replaced by a Dual Chambered Bag 491, which includes an adapter 252 to receive a spiked cannula 255 to put the interior of the dual chambered bag 491 in fluid communication with the secondary medication set or line 260. In this embodiment, the Dual Chambered Bag 491 includes a first side chamber 484 containing medication or concentrated drug and a second side chamber 486; the two side chambers are separated by a Vertical Separation Seal 481. In this embodiment, the Dual Chambered Bag 491 is retro-primed with primary fluid from the Primary Bag 210, as seen with the method illustrated in FIGS. 3a through 3d, filling the second side chamber 486 of the Dual Chambered Bag 491 to a desired fluid volume level as labeled by a range of markings 467 beginning with a minimum of fluid required to allow equally applied pressure to force the Vertical Separation Seal 481 open to mix the retro primed diluent fluid from the second side chamber 486 and the concentrated drug from the first side chamber 484 together for desired mixing. In some embodiments, the Dual Chambered Bag 491 may have the first side chamber 484 filled with powdered antibiotic medication while others may have the first side chamber 484 filled with medication in a liquid concentrate.

The retro-primed medication delivery system described above, in its various embodiments, used in conjunction with the method of retro-priming a medication bag with primary fluid sourced from a primary bag, presents distinct advantages over the systems and methods known in the art for intravenous medication administration.

For example, compared to the method of manually pushing a medication fluid from a syringe, or using a syringe pump to accomplish essentially the same task, the retro-primed medication delivery system generally both allows for a slower, more steady administration of the medication fluid and presents the clinician with a greater range of possible concentrations and fluid volumes for the medication fluid. The manual push process requires that the clinician remain at the patient's bedside until the entire medication dose has been administered. With the present invention, once the medication bag has been retro-primed and the medication mixed with the primary fluid, the clinician is free to allow the medication fluid to flow from the medication bag to the patient; the clinician need not remain with the patient as the medication is administered. With the present invention, there is no danger that a clinician, or a miscalibrated syringe pump, will push the medication too quickly, resulting in pain or other complications for the patient. Instead, the medication fluid flows to the patient under the force of gravity or is controlled by a large-volume IV pump. Further, a syringe usually has a limited fluid volume, which may accommodate less fluid volume than is recommended for the medication being administered. By contrast, with the present invention, the medication can be mixed with a range of fluid volumes to achieve the desired medication concentration and fluid volume. As explained above, the medication bag includes graduated markings, visible from the outside of the bag, to allow a clinician using the bag to determine how much primary fluid has been added to the bag during the retro-priming process. A clinician retro-priming the medication bag is free to select the amount of fluid that is allowed to enter the medication bag, limited only by the maximum fluid capacity of the bag. Thus, for example, while a syringe might only allow for a maximum fluid volume of 10 ml or 50 ml, a clinician retro-priming a 200-ml medication bag has the option to fill the bag with any desired primary fluid volume short of 200 ml. Thus, the patient receives the medication in a less concentrated form and over a greater period of time than is normally possible when a manual IV push or a syringe pump is utilized.

Similarly, the retro-primed medication delivery system described above, in its various embodiments, used in conjunction with the method of retro-priming a medication bag with primary fluid from a primary bag, presents advantages over the use of fluid-filled bags in a piggy-back system. The retro-priming method allows clinicians to mix medications with primary fluid within the sterile IV set. No 797-compliant pharmacy clean room is necessary; nor is an expensive robotic medication admixture system.

Compared to frozen or refrigerated pre-mixed IV medication bags prepared off-site, the retro-priming system reduces the need for storage space and increases the shelf life of medications to be administered intravenously. When a medication is mixed with a fluid in an IV medication bag, the addition of the fluid generally reduces (sometimes substantially) the medication's shelf life, even when the pre-mixed IV medication bag is kept refrigerated. In some embodiments of the present invention, a medication bag arrives at the hospital with the medication bag containing a dry, powdered medication. A medication bag containing dry, unreconstituted medication will almost invariably have a longer shelf life and take up less storage space than pre-mixed IV medication bag, which is already loaded with fluid. A medication bag containing dry medication is also less likely to require refrigeration than a pre-mixed IV medication bag. Further, space and cost considerations limit the number and variety of pre-mixed IV medication bags that a hospital can keep on site. Thus, the available selection at a given hospital of pre-mixed IV medication bags for a particular medication may not include the specific volume of fluid or concentration of medication that a physician requires for a particular patient. So, for example, a clinician desiring to administer “medication X” may find that the pre-mixed IV medication bags are only available in fluid volumes of 100 ml or 200 ml when the clinician in fact would like a fluid volume of 150 ml. The clinician might find that the pre-mixed IV medication bags only include medication X reconstituted with saline solution, when the treatment regimen calls for dextrose solution. The clinician may find that none of the pre-mixed IV medication bags include the desired concentration of medication X. With the present invention, a clinician can take a dry medication bag containing powdered medication X and retro-prime the medication bag with the desired volume of whichever primary fluid is called for, using the graduated markings on the medication bag to see how much primary fluid has filled the medication bag and to calculate the concentration of the now-reconstituted medication X.

The present invention also presents advantages over the burette system described above. For a burette to drain of fluid, air must enter the burette through an air vent. The air vent presents an infection risk, as contaminants and airborne microorganisms may enter the burette chamber through the air vent, therein mixing with the fluid and traveling with the fluid to the patient. By contrast, the present retro-primed medication delivery system is a closed, sterile system; no air vents are necessary to allow the bags to drain of primary fluid and medication fluid. Further, with the burette system, once the burette chamber has emptied of fluid, or is nearly empty, there is a risk that an air bubble will enter the line and pass into the patient, with adverse consequences. Therefore, it is often necessary for a clinician to monitor the burette system closely and then close off the flow of fluid from burette before all of the fluid in the burette chamber has emptied the chamber. Thus, in order to avert the possibility of an air embolism, some of the medication mixed in the fluid often is not delivered to the patient. By contrast, with the present invention, there exists substantially less risk that air will enter the system, even when the medication bag and the primary bag have emptied of fluid. Once the medication bag has been retro-primed, the medication mixed with the primary fluid, and the medication bag put in place, the present system requires less supervision than the burette system.

While the present invention has been illustrated by description of several embodiments and while the illustrative embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

What is claimed is:

1. A method of administering a medication to a patient without interrupting delivery of a primary fluid comprising:
supplying a primary bag containing a primary fluid;
supplying a medication vessel adapted to contain a medication;
connecting the primary bag and the medication vessel to a fluid transfer system, said fluid transfer system being in fluid communication with a delivery device for passing fluid to the patient, said fluid transfer system allowing the passage of fluid between the primary bag, the medication vessel, and the delivery device, said fluid transfer system moving fluid through gravity;
placing a medication in the medication vessel;
positioning the medication vessel in a position relative to the primary bag such that primary fluid from the primary bag moves through said fluid transfer system into the medication bottle, whereby the primary fluid moving into the medication bottle interacts with the medication to form a medication fluid; and
elevating the medication vessel to a higher position relative to the primary bag such that the medication fluid moves from the medication vessel through said fluid transfer system into the delivery device.

2. The method of claim 1 wherein the medication vessel includes pre-marked lines for spotting fluid levels within the medication vessel.

3. The method of claim 1 wherein the medication vessel comprises a medication bottle with pre-marked lines for spotting fluid levels within the medication bottle.

4. The method of claim 1 wherein the medication vessel comprises a dual-chambered bag.

5. The method of claim 1 wherein the medication vessel comprises a dual-chambered bag with an upper chamber containing a medication.

6. The method of claim 1 wherein the medication vessel comprises a dual-chambered bag with a side chamber containing a medication.

7. A system for administering a medication to a patient without interrupting delivery of a primary fluid by preparing a medication fluid that includes primary fluid drawn from the patient's source of primary fluid, the system comprising:
a primary bag containing a primary fluid;
a medication vessel adapted to contain a medication, the medication vessel including graduated markings permitting a person handling the medication vessel to determine by visual inspection the approximate quantity of fluid within the medication vessel;
an IV administration set comprising a primary line in fluid communication with the primary bag, a medication line in fluid communication with the medication vessel, and a principal line connected to both the primary line and the medication line, such that the primary bag and the medication vessel are in fluid communication with each other through the IV administration set, whereby primary fluid from the primary bag is able to pass from the primary bag through the primary line, the principal line, and medication line into the medication vessel; and
an intravenous delivery device in fluid communication with the principal line of the IV administration set, the intravenous delivery device adapted to pass fluid from the IV administration set to the patient;
whereby a practitioner lowers the medication vessel into a position such that primary fluid from the primary bag flows through the IV primary line, the principal line, and the medication line into the medication vessel, where the primary fluid is mixed with medication to form a medication fluid, the practitioner observes the flow of primary fluid into the medication vessel and measuring by visual inspection the fluid level within the medication bottle, and, when the fluid level within the medication vessel has reached a predetermined level, the practitioner elevates the medication vessel into a position such that the primary fluid from the primary bag is no longer flowing into the medication vessel and the medication fluid from the medication bottle flows through the medication line, the principal line, and the intravenous delivery device into the patient, with the medication fluid taking gravimetric precedence over the primary fluid from the primary bag until such time as all of the medication fluid has been administered to the patient.

8. The system of claim 7 wherein the medication vessel includes an adapter to allow a practitioner to insert medication into the medication bottle through a syringe.

9. The system of claim 7 wherein the medication vessel comprises a medication bottle.

10. The system of claim 7 wherein the medication vessel comprises a dual-chambered bag.

11. The system of claim 7 wherein the medication vessel comprises a dual-chambered bag with an upper chamber containing a medication.

12. The system of claim 7 wherein the medication vessel comprises a dual-chambered bag with a side chamber containing a medication.
